(19) United States
(12) Patent Application Publication
(10) Pub. No.: US 2006/0120965 A1
(43) Pub. Date: Jun. 8, 2006

(54) Trimeric, macrocyclically substituted aminoisophthalic acid-halo-benzene derivatives

(76) Inventors: Johannes Platzek, Berlin (DE); Heiko Schirmer, Berlin (DE); Hanns-Joachim Weinmann, Berlin (DE); Jose Luis Martin, Madrid (ES); Juan R. Harto, Madrid (ES)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/274,895

(22) Filed: Nov. 16, 2005

Related U.S. Application Data
(63) Continuation of application No. 11/135,656, filed on May 24, 2005.
(60) Provisional application No. 60/575,417, filed on Jun. 1, 2004.

(30) Foreign Application Priority Data
May 25, 2004 (DE) 102004026103.2

(51) Int. Cl. A61K 49/10; A61K 49/04; C07F 5/00

(52) U.S. Cl. 424/9.363; 424/9.42; 534/16; 540/465

(57) ABSTRACT

The metal complexes of general formula I 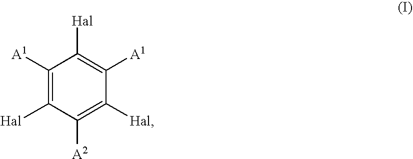
in which Hal stands for bromine or iodine, and A¹ and A² have different meanings, are suitable as contrast media.

This application claims the benefit of the filing date of U S. Provisional Application Ser. No. 60/575,417 filed Jun. 1, 2004 which is incorporated by reference herein.

The invention relates to the subjects that are characterized in the claims: new trimeric, macrocyclically substituted triiodine and tribromobenzene derivatives, their production and use as contrast media in x-ray diagnosis and MRT diagnosis.

During the last decade, impressive advances were achieved in imaging diagnosis. The imaging techniques, such as DAS, CT and MRT, have developed into standard and indispensable tools in diagnosis and interventional radiology and now offer a spatial resolution of less than 1 mm. In addition, the possible applications of these techniques are increased decisively by the use of contrast media. This now wide distribution and acceptance of the contrast media in x-ray diagnosis can be attributed to the introduction of non-ionic monomeric triiodoaromatic compounds in the 1980's, as well as the isoosmolar dimeric iodoaromatic compounds that were introduced in the 1990's. By these two compound classes, the frequency of contrast medium-induced side effects was reduced to 2-4% (Bush, W. H., Swanson, D. P.: Acute Reactions to Intravascular Contrast Media: Types, Risk Factors, Recognition and Specific Treatment. AJR 157 1153-1161, 1991. Rydberg, J., Charles, J., Aspelin, P.: Frequency of Late Allergy-Like Adverse Reactions Following Injection of Intravascular Non-Ionic Contrast Media. Acta Radiologica 39, 219-222, 1998). The use of contrast media in connection with modern imaging techniques now extends from the detection of tumors, for high-resolution vascular visualization, to the quantitative determination of physiological factors such as permeability or perfusion of organs. The concentration of the x-ray contrast medium (here the iodine atom) is decisive for the contrast and the detection sensitivity. Despite further development of the technology, it was not possible to reduce the concentration or the dose to be administered that is necessary for a medical diagnosis. Thus, in a standard CT study, 100 g of substance or more is injected per patient.

Although the compatibility of the x-ray contrast media has been improved by the introduction of non-ionic triiodobenzenes, the number of side effects is still always high. Because of very high study numbers of several million per year in x-ray diagnosis, ten thousand patients are thus affected. These contrast medium-induced side effects extend from slight reactions such as nausea, dizziness, vomiting, and hives up to severe reactions such as bronchial spasms, or renal failure up to reactions such as shock or even death. Fortunately, these severe cases are very rare and are observed at a frequency of only 1/200,000 (Morcos, S. K., Thomsen, H. S.: Adverse Reactions to Iodinated Contrast Media. Eur Radiol 11, 1267-1275, 2001).

The frequency of these side effects, which are also observed as pseudoallergic contrast medium-induced side effects, is, however, increased by about a factor of 3 in atopic patients and by a factor of 5 in patients with a previous history of contrast medium-induced side effects. Asthma increases the risk of severe contrast medium-induced side effects by a factor of 6 in non-ionic contrast media (Thomsen, H. S., Morcos, S. K.: Radiographic Contrast Media. BJU 86 (Suppl1), 1-10, 2000. Thomsen, H. S., Dorph, S.: High-Osmolar and Low-Osmolar Contrast Media. An Update on Frequency of Adverse Drug Reactions. Acta Radiol 34, 205-209, 1993. Katayama, H., Yamaguchi, K., Kozuka, T., Takashima, T., Seez, P., Matsuura, K.: Adverse Reactions to Ionic and Non-Ionic Contrast Media. Radiology 175, 621-628, 1990. Thomsen, H. S., Bush, Jr., W. H.: Adverse Effects on Contrast Media. Incidence, Prevention and Management. Drug Safety 19: 313-324, 1998). Under these conditions, the examiners for x-ray diagnosis in recent years most frequently use non-iodine-containing Gd-chelates instead of the standard triiodoaromatic compounds in computer topography but also in interventional radiology as well as DSA (Gierada, D. S., Bae, K. T.: Gadolinium as CT Contrast Agent: Assessment in a Porcine Model. Radiology 210, 829-834, 1999. Spinosa, D. J., Matsumoto, A. H., Hagspiel, K. D., Angle, J. F., Hartwell, G. D.: Gadolinium-based Contrast Agents in Angiography and Interventional Radiology. AJR 173; 1403-1409, 1999. Spinosa, D. J., Kaufmann, J. A., Hartwell, G. D. : Gadolinium Chelates in Angiography and Interventional Radiology: A Useful Alternative to Iodinated Contrast Media for Angiography. Radiology 223, 319-325, 2002). This is, on the one hand, substantiated by the very good compatibility of the metal chelates that are used in MRT, but also by the known fact that lanthanides are also x-ray-opaque. In comparison to iodine, gadolinium and other lanthanides show a greater absorption than iodine especially at higher voltages/energies of the x-ray radiation, such that, in principle, they are suitable as opacifying elements for x-ray diagnosis (Schmitz, S., Wagner, S., Schuhmann-Giampieri, G., Wolf, K. J.: Evaluation of Gadobutrol in a Rabbit Model as a New Lanthanide Contrast Agent for Computer Tomography. Invest. Radiol. 30(11): 644-649, 1995).

The above-mentioned Gd-containing chelate compounds originally used in the MRT are also readily water-soluble and are distinguished by an excellent compatibility. Compared to the iodine-containing/non-ionic contrast media, the rate of light pseudoallergenic reactions is greatly reduced, and the rate of fatal reactions is extremely rare and is indicated with 1/1,000,000 (Runge, V. M.: Safety of Approved MR Contrast Media for Intravenous Injection. J. Magn Reson Imaging 12, 205-213, 2000). In contrast to other contrast medium-induced side effects, such as, e.g., the renal compatibility, pseudoallergic reactions are more likely independent of the administered dose. Also, the smallest dosages can accordingly already trigger a pseudoallergic reaction.

Desired are substances that combine the advantages of the two chemically entirely different classes of compounds.

The extraordinarily high hydrophilia of the metal chelates suggests a low incompatibility rate. Iodoaromatic compounds have a higher lipophilia by a factor of 100-200 (larger distribution coefficient between butanol/water) than metal chelates.

Based on the low substance concentration and the low specific proportion of the imaging metal in the entire molecule, the previously known metal chelates for x-ray diagnosis are not optimal (Albrecht, T., Dawson, P.: Gadolinium-DTPA as X-ray Contrast Medium in Clinical Studies. BJR 73, 878-882, 2000). More recent attempts to solve this problem describe the production of metal complex conjugates, in which triiodoaromatic compounds are covalently bonded to an open-chain or macrocyclic metal complex (U.S. Pat. No. 5,324,503, U.S. Pat. No. 5,403,576, WO 93/16375, WO 00/75141, WO 97/01359, WO 00/71526, U.S. Pat. No. 5,660,814). Because of their low hydrophilia and high viscosity, the latter cannot be administered in adequate concentration and reasonable volumes, however.

The purpose is to produce compounds that have an adequate hydrophilia—comparable to that of Gd-chelates—and in addition to exhibit a high concentration of opacifying elements. Values that are significantly higher than those in metal chelates, which are approximately 25% (g/g), were desirable. In addition, at a higher concentration, a very good water solubility must be provided. In addition to their good pharmacological properties, the highly concentrated solutions must also indicate a practical viscosity and a low osmotic pressure.

This object is achieved by this invention.

1. The metal complexes of general formula I according to the invention 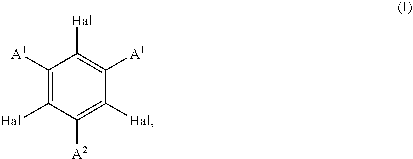
in which

Hal stands for bromine or iodine,

A¹ stands for the radical —CONH_13 (CH₂)₂—NH—CO—CH(CH₃)—K

A² stands for the radical —N(CH₃)—CO—CH₂—NH—CO—CH(CH₃)—K,

K stands for a macrocyclic compound of formula I_{A} 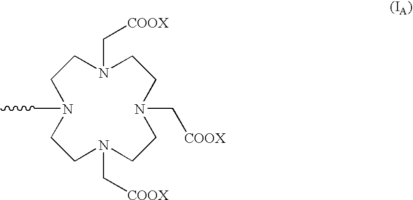
with X in the meaning of a hydrogen atom or a metal ion equivalent of atomic numbers 20-29, 39, 42, 44 or 57-83, provided that at least two X stand for metal ion equivalents and optionally present free carboxy groups optionally are present as salts of organic and/or inorganic bases or amino acids or amino acid amides, show a very good solubility and a distribution coefficient that is comparable to that of Gd-chelates. In addition, the new compounds have a high specific content of opacifying elements, a low viscosity and osmolality and thus good tolerance/compatibility, so that they are extremely well suited as contrast media for x-ray and MR imaging.

The compounds of general formula I according to the invention can be produced according to the process that is known according to one skilled in the art by a triiodo- or tribromoaromatic compound of general formula II 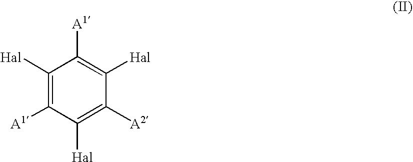
being reacted in a way that is known in the art with a macrocyclic compound of general formula III 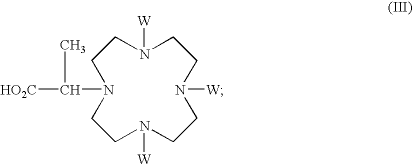
in which

W stands for a protective group, A^{1′} in the meaning of —CO—NH—(CH₂)₂—NH₂

and A² in the meaning of —N(CH₃)—CO—CH₂—NH₂

and then protective group W being removed and the radical CH₂COOX being introduced in a way that is known in the art and then reacted in a way that is known in the art with a metal oxide or metal salt of an element of atomic numbers 20-29, 39, 42, 44 or 57-83.

As amino protective groups W, the benzyloxycarbonyl, tert-butoxycarbonyl, trifluoroacetyl, fluorenylmethoxycarbonyl, benzyl, formyl, 4-methoxybenzyl, 2,2,2-trichloroethoxycarbonyl, phthaloyl, 1,2-oxazoline, tosyl, dithiasuccinoyl, allyloxycarbonyl, sulfate, pent-4-enecarbonyl, 2-chloroacetoxymethyl (or ethyl) benzoyl, tetrachlorophthaloyl, and alkyloxycarbonyl groups that are familiar to one skilled in the art can be mentioned [Th. W. Greene, P. G. M. Wuts, Protective Groups in Organic Syntheses, 2nd Ed., John Wiley and Sons (1991), pp. 309 - 385; E. Meinjohanns et al, J. Chem. Soc. Pekin Trans 1, 1995, 405; U. Ellensik et al, Carbohydrate Research 280, 1996, 251; R. Madsen et al, J. Org. Chem. 60, 1995, 7920; R. R. Schmidt, Tetrahedron Letters 1995, 5343].

The cleavage of the protective groups is carried out according to the process that is known to one skilled in the art (see, e.g., E. Wünsch. Methoden der Org. Chemie [Methods of Organic Chemistry], Houben-Weyl, Vol. XV/1, 4^{th} Edition 1974, p. 315), for example by hydrolysis, hydrogenolysis, alkaline saponification of esters with alkali in aqueous-alcoholic solution at temperatures from 0° C. to 50° C., acidic saponification with mineral acids, or in the case of Boc groups with the aid of trifluoroacetic acid.

The introduction of the desired metal ions can be carried out as was disclosed in Patents EP 71564, EP 130934 and DE-OS 34 01 052. To this end, the metal oxide or a metal salt (for example, a chloride, nitrate, acetate, carbonate or sulfate) of the desired element is dissolved or suspended in water and/or a lower alcohol (such as methanol, ethanol or isopropanol) and reacted with the solution or suspension of the equivalent amount of the complexing agent.

The neutralization of optionally still present free carboxy groups is carried out with the aid of inorganic bases (e.g., hydroxides, carbonates or bicarbonates) of, e.g., sodium, potassium, lithium, magnesium or calcium and/or organic bases, such as, i.a., primary, secondary and tertiary amines, such as, e.g., ethanolamine, morpholine, glucamine, N-methyl- and N,N-dimethylglucamine, as well as basic amino acids, such as, e.g., lysine, arginine, and ornithine or amides of original neutral or acidic amino acids.

For the production of neutral complex compounds, for example in acidic complex salts in aqueous solution or suspension, enough of the desired base can be added to reach the neutral point. The solution that is obtained can then be evaporated to the dry state in a vacuum. It is frequently advantageous to precipitate the neutral salts that are formed by adding water-miscible solvents, such as, e.g., lower alcohols (methanol, ethanol, isopropanol, etc.), lower ketones (acetone, etc.), or polar ethers (tetrahydrofuran, dioxane, 1,2-dimethoxyethane, etc.) and thus to obtain easily isolated and readily purified crystallizates. It has proven especially advantageous to add the desired base as early as during the complexing of the reaction mixture and thus to save a process step.

The purification of the thus obtained complexes is carried out, optionally after the pH is set to 6 to 8, preferably about 7, by adding an acid or base, preferably by ultrafiltration with membranes of a suitable pore size (e.g., Amicon®YM1, Amicon®YM3), gel filtration on, e.g., suitable Sephadex® gels or by HPLC on silica gel or reverse-phase material.

A purification can also be carried out by crystallization from solvents such as methanol, ethanol, i-propanol, acetone or their mixtures with water.

In the case of neutral complex compounds, it is frequently advantageous to add the oligomer complexes via an anion exchanger, for example IRA 67 (OH⁻ form), and optionally in addition via a cation exchanger, for example IRC 50 (H⁺ form), to separate ionic components.

The production of the compounds of general formula I according to the invention can be carried out as indicated above:

The reaction of triiodo- or tribromoaromatic compounds of general formula II with compounds of general formula III is carried out according to the process of amide formation that is known to one skilled in the art.

In this connection, either a direct coupling of the free acid of III with the free amine of II can be performed with dehydrating reagents, such as dicyclohexyl-carbodiimide, diisopropylcarbodiimide, EDC, EEDQ, TBTU, or HATU in aprotic solvents such as DMF, DMA, THF, dioxane, toluene, chloroform or methylene chloride at temperatures of 0°-50° C., or else the acid group is activated in the compound of general formula III, by its first being converted into an active ester and then these esters into a solvent, such as, for example, DMF, DMA, THF, dioxane, dichloromethane, i-ProOH, or toluene, optionally with the addition of an organic or inorganic base, such as NTEt₃, pyridine, DMAP, Hünig base, Na₂CO₃, or CaCO₃, being reacted at temperatures of −10° to +70° C. with the amine of general formula II.

Activated carboxyl groups are defined as those carboxyl groups that are derivatized such that they facilitate the reaction with an amine. Which groups can be used for activation is known, and reference can be made to, for example, M. and A. Bodanszky, “The Practice of Peptide Synthesis”, Springerverlag 1984. Examples are aducts of carboxylic acid with carbodiimides or activated esters, such as, e.g., hydroxybenzotriazole ester, acid chloride, N-hydroxysuccinimide ester,

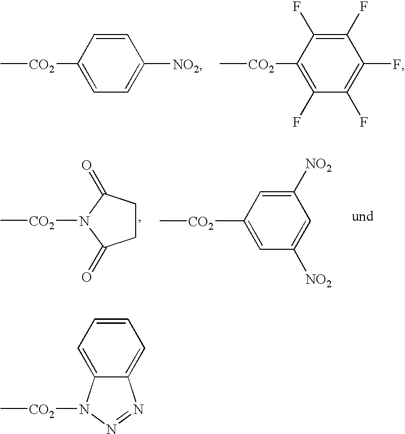

[and] 4-Nitrophenyl ester and N-hydroxysuccinimide ester are preferred.

The activated esters of the above-described compounds are produced as known to one skilled in the art. Also, the reaction with correspondingly derivatized esters of N-hydroxysuccinimide, such as, for example: 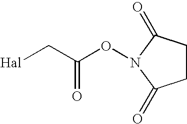
is possible (Hal=halogen).

In general, for this purpose, all commonly used activating methods for carboxylic acids can be used that are known in the prior art. The activation of the carboxylic acid is carried out according to commonly used methods. Examples of suitable activating reagents are dicyclohexylcarbodiimide (DCC), 1-ethyl-3-(3-dimethylaminopropyl)-carbodiimide-hydrochloride (EDC), benzotriazol-1-yloxytris(dimethylamino)-phosphonium hexafluorophosphate (BOP) and O-(benzotriazol-1-yl)-1,1,3,3-tetramethyl uronium hexafluorophosphate (HBTU), preferably DCC. Also, the addition of O-nucleophilic catalysts, such as, e.g., N-hydroxysuccinimide (NHS) or N-hydroxybenzotriazole, is possible.

Advantageously used as nucleofuges are the radicals:

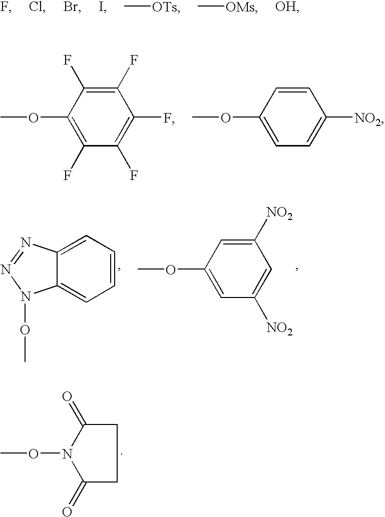

The production of compound II is described in the examples.

The production of the corresponding tri-bromine compound is carried out analogously to what is described in EP 0073715.

Compounds of general formula III are described in, e.g., WO 97/0205 1, WO 99/16757 or can be produced simply from tri-Boc cyclene or tri-Z-cyclene according to methods that are known in the literature.

The compounds according to the invention can be used both in x-ray diagnosis and in MR diagnosis.

The high x-ray opacity paired with the good water-solubility thereof of the halogenated x-ray contrast media is combined with the intense hydrophilia of metal chelates and good compatibility in a molecule that is inherent in them. The very high hydrophilia of the new compounds results in that the side-effect profile corresponds to that of the very well-tolerated Gd compounds, as they are used in MR imaging. This property therefore makes it especially suitable for use in patients with a proven allergy to iodized compounds or in the case of existing atopy. In particular, the incidence of severe side effects such as bronchial spasms and shock or even death is reduced to the low level of the MR contrast medium.

The low osmolality of the formulations is an indication of a generally very good compatibility of the new compounds. They are therefore especially suitable for intravascular (parenteral) uses.

Depending on the pharmaceutical formulation, the contrast media can be used exclusively for x-ray diagnosis (trihalogen complexes with diamagnetic metals), but also simultaneously for x-ray diagnosis and MRT diagnosis (trihalogen complexes with paramagnetic atoms, preferably Gd). The compounds can very advantageously be used in, e.g., urography, computer tomography, angiography, gastrography, mammography, cardiology and neuroradiology. Even in the case of radiation therapy, the complexes that are used are advantageous. The compounds are suitable for all perfusion measurements. A differentiation of areas that are well supplied with blood and ischemic areas is possible after intravascular injection. Quite generally, these compounds can be used in all indications where conventional contrast media are used in x-ray diagnosis or MR diagnosis.

The new contrast media can also be used for the magnetization-transfer technique (see, e.g., Journ. Chem. Phys. 39(11), 2892 (1963), as well as WO 03/013616), if they contain mobile protons in their chemical structure.

The contrasting of cerebral infarctions and tumors of the liver or space-occupying processes in the liver as well as of tumors of the abdomen (including the kidneys) and the muscle-skeleton system is especially valuable diagnostically. Based on the low osmotic pressure, the blood vessels can be visualized especially advantageously after intraarterial or else intravenous injection.

If the compound according to the invention is intended for use in MR diagnosis, the metal ion of the signaling group must be paramagnetic. These are in particular the divalent and trivalent ions of the elements of atomic numbers 21-29, 42, 44 and 58-70. Suitable ions are, for example, the chromium(III), iron(II), cobalt(II), nickel(II), copper(II), praseodymium(III), neodymium(III), samarium(III) and ytterbium(III) ion. Because of their strong magnetic moment, gadolinium(III), terbium(III), dysprosium(III), holmium(III), erbium(III), iron(III) and manganese(II) ions are preferred; gadolinium(III) and manganese(II) ions are especially preferred.

If the compound according to the invention is intended for use in x-ray diagnosis, the metal ion is preferably derived from an element of a higher atomic number to achieve an adequate absorption of the x-rays. It was found that for this purpose, diagnostic agents that contain a physiologically compatible complex salt with metal ions of elements of atomic numbers 25, 26 and 39 as well as 57-83 are suitable.

Manganese(II), iron(II), iron(III), praseodymium(III), neodymium(III), samarium(III), gadolinium(III), ytterbium(III) or bismuth(III) ions, especially dysprosium(III) ions and yttrium(III) ions, are preferred.

The production of the pharmaceutical agents according to the invention is carried out in a way that is known in the art by the complex compounds according to the invention—optionally with the addition of the additives that are commonly used in galenicals—being suspended or dissolved in aqueous medium and then the suspension or solution optionally being sterilized. Suitable additives are, for example, physiologically harmless buffers (such as, for example, tromethamine), additives of complexing agents or weak complexes (such as, for example, diethylenetriaminepentaacetic acid or the Ca complexes that correspond to the metal complexes according to the invention) or—if necessary—electrolytes such as, for example, sodium chloride, or—if necessary—antioxidants, such as, for example, ascorbic acid.

If suspensions or solutions of the agents according to the invention in water or physiological salt solution are desired for enteral or parenteral administration or other purposes, they are mixed with one or more adjuvant(s) that are commonly used in galenicals [for example, methyl cellulose, lactose, mannitol] and/or surfactant(s) [for example, lecithins, Tween®, Myrj®] and/or flavoring substance(s) for taste correction [for example, ethereal oils].

In principle, it is also possible to produce the pharmaceutical agents according to the invention without isolating the complexes. In any case, special care must be taken to perform the chelation so that the complexes according to the invention are virtually free of noncomplexed metal ions that have a toxic effect.

This can be ensured, for example, with the aid of color indicators such as xylenol orange by control titrations during the production process. The invention therefore also relates to the process for the production of complex compounds and their salts. As a final precaution, there remains purification of the isolated complex.

In the in-vivo administration of the agents according to the invention, the latter can be administered together with a suitable vehicle, such as, for example, serum or physiological common salt solution and together with another protein such as, for example, human serum albumin (HSA).

The agents according to the invention are usually administered parenterally, preferably i.v. They can also be administered intraarterially or interstitially/intracutaneously, depending on whether a vessel/organ is to be visualized selectively contrasted (e.g., visualization of the coronary arteries after intraarterial injection) or tissue or pathologies (e.g., diagnosis of cerebral tumors after intravenous injection).

The pharmaceutical agents according to the invention contain preferably 0.001-1 mol/l of the above-mentioned compound and are generally dosed in amounts of 0.001-5 mmol/kg.

The agents according to the invention meet the many requirements for suitability as contrast media for magnetic resonance tomography. After oral or parenteral administration by increasing the signal intensity, they are extremely well suited for enhancing the informational value of the image that is obtained with the aid of an MR tomograph. They also show the high effectiveness that is necessary to load the body with the minimum possible amounts of foreign substances and the good compatibility that is necessary to maintain the non-invasive nature of the studies. The high effectiveness (relaxivity) of the paramagnetic compounds according to the invention is of great advantage for use in magnetic resonance tomography. Thus, the relaxivity (L/mmol⁻¹.sec⁻¹ of gadolinium-containing compounds is generally 2 to 4×greater than in conventional Gd complexes (e.g., gadobutrol).

The good water solubility and low osmolality of the agents according to the invention makes it possible to produce highly concentrated solutions, so as to keep the volume burden of the circulatory system within reasonable limits and to offset the dilution by bodily fluids. In addition, the agents according to the invention exhibit not only high stability in-vitro, but also surprisingly high stability in-vivo, so that a release or an exchange of the ions, which are inherently toxic and are bonded in the complexes, is carried out only extremely slowly within the time that it takes for the new contrast media to be completely excreted.

In general, the agents according to the invention are dosed for use as MRT diagnostic agents in amounts of 0.001-5;mmol of Gd/kg, preferably 0.005 - 0.5 mmol of Gd/kg.

The agents according to the invention are extremely well suited as x-ray contrast media, whereby it is especially to be emphasized that with them, no signs of the anaphylaxis-like reactions that are known from the iodine-containing contrast media can be detected in biochemical-pharmacological studies. In the case of strong x-ray absorption, they are especially effective in areas of higher tube voltages (e.g., CT and DSA).

In general, the agents according to the invention are dosed for administration as x-ray contrast media analogously to, for example, meglumine-diatrizoate, in amounts of 0.01-5 mmol/kg, preferably 0.02-1 mmol of substance/kg, which corresponds to 0.06-6 mmol (I+Dy)/kg in the case of, e.g., iodine-Dy compounds. Depending on the diagnostic requirement, formulations can be selected that can be used both in x-ray diagnosis and in MR diagnosis. To achieve optimal results for both imaging modalities, it may be advantageous to select formulations in which the proportion of paramagnetic ions is reduced, since for many MR diagnostic applications, a point of diminishing returns is reached with too high a proportion of paramagnetic ions.

For dual uses, formulations can be used in which the proportion, in percent, of paramagnetic substances (e.g., Gd) is reduced to 0.05 to 50, preferably to 2-20%. As an example, a cardiac diagnostic application can be mentioned. For the examination, a formulation that consists of the substances according to the invention in a total concentration of, e.g., 0.25 mol/l is used. The proportion of Gd-containing complexes is 20%, the remaining 80% of the metals are, e.g., Dy atoms. In an x-ray coronary angiography after intraarterial or intravenous administration, e.g., 50 ml is used, i.e., 0.18 mmol of substance per kg of body weight in a patient who weighs 70 kg. Shortly after x-ray visualization of the coronary vessels has taken place, an MR diagnosis of the heart is followed to be able to differentiate vital myocardial areas from necrotic myocardial areas. The amount of about 110 μmol of Gd/kg previously administered for the test is optimal for this purpose.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### PATENT EXAMPLES

### Example 1

### a) 2,4,6-Triiodo-5-{methyl[2-(2,2,2-trifluoroacetylamino)acetyl]amino}isophthalic acid dichloride

14.5 ml (200 mmol) of thionyl chloride is added in drops at 0° C. within 1 hour to a solution of 34.2 g (200 mmol) of glycine trifluoroacetate in 200 ml of dimethylacetamide. Then, 24.4 g (40 mmol) of 5-amino-2,4,6-triiodoisophthalic acid dichloride (EP 0033426, Sovak, 1/80 US) is added at 0° C. and stirred for 4 days at room temperature. The reaction mixture is poured into 5 liters of ice water, and the solid that accumulates is filtered off. For further purification, the filter residue is dissolved in 1000 ml of ethyl acetate, shaken out twice with saturated sodium bicarbonate solution, the organic phase is dried on sodium sulfate, and the solvent is concentrated by evaporation in a vacuum.

Yield: 28.7 g (94% of theory) of a colorless solid

Elementary analysis:

Cld.: C, 20.47; H, 0.79; N, 3.67.

Fnd.: C, 20.52; H, 0.77; N, 3.71.

### b) 5-[(2-Aminoacetyl)methylamino)-N,N-bis-(2-aminoethyl)-2,4,6-triiodoisophthalic acid amide

A solution of 10 g (13.1 mmol) of 2,4,6-triiodo-5- {methyl-[2-(2,2,2-trifluoroacetylamino)-acetyl]amino}isophthalic acid dichloride in 100 ml of tetrahydrofuran is added in drops to 26.7 ml (399 mmol) of ethylenediamine over 1 hour at room temperature, and it is stirred for 14 more hours. The precipitated solid is filtered off, rewashed with ethanol, taken up in 100 ml of water, and the pH is set at 8.0 with 1 M lithium hydroxide solution. After concentration by evaporation in a vacuum, it is recrystallized from ethanol.

Yield: 7.3 g (78% of theory) of a colorless solid

Elementary analysis:

Cld.: C, 25.23; H, 2.96; N, 11.77; I, 53.31.

Fnd.: C, 25.44; H, 2.98; N, 11.81; I, 53.09.

### c) 1,4,7-Tris-(benzyloxycarbonyl)-10-(1-ethoxycarbonylethyl)-1,4,7,10-tetraazacyclo-dodecane

50.1 g (87.0 mmol) of 1,4,7-tris-(benzyloxycarbonyl)-1,4,7,10-tetraazacyclododecane (Delaney et al., J. Chem. Soc. Perkin Trans. 1991, 3329) is dissolved in 500 ml of acetonitrile and mixed with 55.5 g (400 mmol) of sodium carbonate. Then, while being stirred vigorously, 54.3 g (300 mmol) of 1-bromopropionic acid ethyl ester is added, and it is heated for 20 hours to 60° C. Insoluble components are filtered out, evaporated to the dry state, and chromatographed on silica gel (mobile solvent ethyl acetate/hexane 20:1). The fractions that contain the product are combined and concentrated by evaporation.

Yield: 46 g (78% of theory) of a colorless oil.

Elementary analysis:

Cld.: C, 65.86; H, 6.87; N, 8.30.

Fnd.: C, 65.99; H, 6.88; N, 8.23.

### d) 1,4,7-Tris-(benzyloxycarbonyl)-10-(1-carboxyethyl)-1,4,7,10-tetrazacyclododecane

33.7 g (50 mmol) of 1,4,7-tris-(benzyloxycarbonyl)-10-(1-ethoxycarbonylethyl)-1,4,7,10-tetrazacyclododecane is dissolved [in] 300 ml of dioxane and mixed with 140 ml of 5% aqueous NaOH solution and stirred for 24 hours at room temperature. After neutralization with concentrated HCl, it is evaporated to the dry state. The residue is taken up in 250 ml of ethyl acetate and extracted twice with 250 ml each of 1N HCl solution. The organic phase is dried on sodium sulfate, and the solvent is evaporated to the dry state.

Yield: 28.2 g (87% of theory) of a colorless solid

Elementary analysis:

Cld.: C, 65.00; H, 6.55; N, 8.66.

Fnd.: C, 65.22; H, 6.59; N, 8.60.

### e) 2,4,6-Triiodo-5-(3-aza-5-methyl-1,4-dioxopentane-1,5-diyl-{10-[1,4,7-tris-(benzyloxycarbonyl)-1,4,7,10-tetraazacyclododecanyl]})methylaminoisophthalic acid-N,N-bis-(3-aza-5-methyl-4-oxopentane-1,5-diyl-{10-[1,4,7-tris-(benzyloxycarbonyl)-1,4,7,1 0-tetraazacyclododecanyl]})amide

109 g (168.5 mmol) of 1,4,7-tris-(benzyloxycarbonyl)-10-(1-carboxyethyl)-1,4,7,10-tetrazacyclododecane, 50 ml (390 mmol) of triethylamine, 34.9 g (168.4 mmol) of dicyclohexylcarbodiimide and 19.4 g (168.4 mmol) of N-hydroxysuccinimide are added to a suspension of 40.0 g (56.0 mmol) of 5-[(2-aminoacetyl)methylamino]-N,N-bis-(2-aminoethyl)-2,4,6-triiodisophthalic acid amide in 1000 ml of DMF, and it is stirred for 20 hours at room temperature. Insoluble components are filtered out and evaporated to the dry state. The residue is taken up in 1000 ml of ethyl acetate and extracted twice with 500 ml each of water. The organic phase is dried on sodium sulfate, the solvent is evaporated to the dry state, and the residue is chromatographed on silica gel (mobile solvent dichloromethane/methanol 20:1). The fractions that contain the product are combined and concentrated by evaporation.

Yield: 80.2 e (55% of theory) of a colorless solid

Elementary analysis:

Cld.: C, 54.43; H, 5.47; N, 9.70; I, 14.64.

Fnd.: C, 54.67; H, 5.42; N, 9.69; I, 14.59.

### f) 2,4,6-Triiodo-5-(3-aza-5-methyl-1,4-dioxopentane-1,5-diyl-{10-[1,4,7,10-tetraazacyclododecanyl]})methylaminoisophthalic acid-N,N-bis-(3-aza-5-methyl-4-oxopentane-1,5-diyl-{10-[1,4,7,10-tetraazacyclododecanyl]})amide

78 g (30 mmol) of 2,4,6-triiodo-5-(3-aza-5-methyl-1,4-dioxopentane-1,5-diyl-{10-[1,4,7-tris-(benzyloxycarbonyl)-1,4,7,10-tetraazacyclododecanyl]})methylaminoisophthalic acid-N,N-bis-(3-aza-5-methyl-4-oxopentane-1,5-diyl-{10-[1,4,7-tris-(benzyloxycarbonyl)-1,4,7,10-tetraazacyclododecanyl]})amide is carefully mixed at 0-5° C. with 500 ml of HBr/AcOH (33%) and stirred for 3 hours at room temperature. Then, the reaction mixture is poured into 2500 ml of diethyl ether, the solid that accumulates in this case is suctioned off and rewashed several times with diethyl ether. The residue is dissolved in 300 ml of water and 300 ml of dichloromethane while being stirred vigorously, and 32% NaOH solution is added until a pH of 10 is reached. The organic phase is separated, the aqueous phase is extracted three times with 150 ml of dichloromethane each, and the combined organic phases are dried on magnesium sulfate and evaporated to the dry state.

Yield: 40.5 g (97% of theory) of a colorless solid

Elementary analysis:

Cld.: C, 41.39; H, 6.30; N, 18.10; I, 27.33.

Fnd.: C, 40.50; H, 6.31; N, 18.07; I, 27.22.

### g) 2,4,6-Triiodo-5-(3-aza-5-methyl-1,4-dioxopentane-1,5-diyl- {10-[1,4,7-tris-(carboxymethyl)-1,4,7,10-tetraazacyclododecanyl]})methylaminoisophthalic acid-N,N-bis-(3-aza-5-methyl-4-oxopentane-1,5-diyl-{10-[1,4,7-tris-(carboxymethyl)-1,4,7,10-tetraazacyclododecanyl]})amide

40 g (28.7 mmol) of 2,4,6-triiodo-5-(3-aza-5-methyl-1,4-dioxopentane-1,5-diyl-{10-[1,4,7,10-tetraazacyclododecanyl]})methylaminoisophthalic acid-N,N-bis-(3-aza-5-methyl-4-oxopentane-1,5-diyl-{10-[1,4,7,10-tetraazacyclododecanyl]})amide is dissolved in 200 ml of water, 41.5 g (439.8 mmol) of chloroacetic acid is added, and a pH of 9.5 is set at 60° C. with 32% NaOH. It is heated for 10 hours to 70° C., whereby the pH of the reaction mixture is continuously readjusted to 9.5. After cooling to room temperature, a pH of 1 is set with concentrated HCl, and the solution is concentrated by evaporation in a vacuum. The residue is absorptively precipitated with 500 ml of methanol, insoluble components are filtered out, and the filtrate is concentrated by evaporation. The residue is dissolved in 200 ml of water and added to an ion-exchange column (1200 ml, IR 120, H⁺-form). Then, it is washed with 5 1 of water, and the acid eluate is concentrated by evaporation. The residue is dissolved in 150 ml of methanol and added in drops in 2500 ml of diethyl ether, the solid that accumulates in this case is suctioned off, rewashed several times with diethyl ether and dried in a vacuum.

Yield: 38 g (69% of theory) of a colorless solid

Elementary analysis:

Cld.: C, 41.39; H, 5.53; N, 13.16; I, 19.88.

Fnd.: C, 41.62; H, 5.57; N, 13.08; I, 19.65.

### h) 2,4,6-Triiodo-5-(3-aza-5-methyl-1,4-dioxopentane-1,5-diyl-{10-1,4,7-tris-(carboxylatomethyl)-1,4,7,10-tetraazacyclododecanyl, Gd-complex]})methylaminoisophthalic acid-N,N-bis-(3-aza-5-methyl-4-oxopentane-1,5-diyl-{10-[1,4,7-tris-(carboxylatomethyl)-1,4,7,10-tetraaza-cyclododecanyl, Gd-complex]})amide

13.2 g (6.9 mmol) of 2,4,6-triiodo-5-(3-aza-5-methyl-1,4-dioxopentane-1,5-diyl-{10-[1,4,7-tris-(carboxymethyl)-1,4,7,10-tetraazacyclododecanyl]})methylaminoiso-phthalic acid-N,N-bis-(3-aza-5-methyl-4-oxopentane-1,5-diyl-{10-[1,4,7-tris-(carboxymethyl)-1,4,7,10-tetraazacyclododecanyl]})amide is dissolved in 100 ml of water and acidified by adding 3 ml of acetic acid. 3.7 g (10.4 mmol) of gadolinium oxide is added and refluxed for 6 hours. After the complexing is completed, it is set at a pH of 7.4 with ammonia and chromatographed on silica gel (mobile solvent: dichloromethane/methanol/ammonia: 10/10/1). The fractions that contain the product are combined and absorptively precipitated with 10 g of ion exchanger (IR 267 H-form) for 2 hours and filtered off, then absorptively precipitated with 10 g of ion exchanger (IRA 67 OH-form) for 2 hours, filtered off, mixed with 2 g of activated carbon, heated for 2 hours to 60° C., filtered off and freeze-dried.

Yield: 9.9 g (56% of theory) of a colorless solid

Water content (Karl-Fischer): 7.1;%

Elementary analysis (relative to the anhydrous substance):

Cld.: C, 33.34; H, 4.07; N, 10.60; I, 16.01; Gd, 19.84.

Fnd.: C, 33.51; H, 4.11; N, 10.65; I, 15.99; Gd, 19.73.

### Example 2

### 2,4,6-Triiodo-5-(3-aza-5-methyl-1,4-dioxopentane-1,5-diyl-{10-[1,4,7-tris-(carboxylatomethyl)-1,4,7,10-tetraazacyclododecanyl, Dy-complex]})methylaminoisophthalic-acid-N,N-bis-(3-aza-5-methyl-4-oxopentane-1,5-diyl-{10-[1,4,7-tris-(carboxylatomethyl)-1,4,7,10-tetraazacyclododecanyl, Dy-complex]})amide

13.2 g (6.9 mmol) of 2,4,6-triiodo-5-(3-aza-5-methyl-1,4-dioxopentane-1,5-diyl-{10-[1,4,7-tris-(carboxymethyl)-1,4,7,10-tetraazacyclododecanyl]})methylaminoiso-phthalic acid-N,N-bis-(3-aza-5-methyl-4-oxopentane-1,5-diyl-{10-[1,4,7-tris-(carboxymethyl)-1,4,7,10-tetraazacyclododecanyl]})amide (title compound 1 g) is dissolved in 100 ml of water and acidified by adding 3 ml of acetic acid. 3.9 g (10.4 mmol) of dysprosium oxide is added, and it is refluxed for 6 hours. After the complexing is completed, it is set at a pH of 7.4 with ammonia and chromatographed on silica gel (mobile solvent: dichloromethane/methanol/ammonia: 10/10/1). The fractions that contain the product are combined and absorptively precipitated with 10 g of ion exchanger (IR 267 H-form) for 2 hours and filtered off, then absorptively precipitated with 10 g, of ion exchanger (IRA 67 OH-form) for 2 hours, filtered off, mixed with 2 g of activated carbon, heated for 2 hours to 60° C., filtered off and freeze-dried.

Yield: 9.4 g (53% of theory) of a colorless solid

Water content (Karl-Fischer): 6.7%

Elementary analysis (relative to the anhydrous substance):

Cld.: C, 33.12; H, 4.04; N, 10.53; I, 15.90; Dy, 20.36.

Fnd.: C, 33.26; H, 4.08; N, 10.55; I, 15.87; Dy, 20.27.

### Example 3

### 2,4,6-Triiodo-5-(3-aza-5-methyl-1,4-dioxopentane-1,5-diyl-{10-[1,4,7-tris-(carboxylatomethyl)-1,4,7,10-tetraazacyclododecanyl, Yb-complex]})methylaminoisophthalic acid-N,N-bis-(3-aza-5-methyl-4-oxopentane-1,5-diyl-{10-[1,4,7-tris-(carboxylatomethyl)-1,4,7,10-tetraazacyclododecanyl, Yb-complex]})amide

13.2 g (6.9 mmol) of 2,4,6-triiodo-5-(3-aza-5-methyl-1,4-dioxopentane-1,5-diyl-{10-[1,4,7-tris-(carboxymethyl)-1,4,7,10-tetraazacyclododecanyl]})methylaminoiso-phthalic acid-N,N-bis-(3-aza-5-methyl-4-oxopentane-1,5-diyl-{10-[1,4,7-tris-(carboxymethyl)-1,4,7,10-tetraazacyclododecanyl]})amide (title compound 1 g) is dissolved in 100 ml of water and acidified by adding 3 ml of acetic acid. 4.1 g (10.4 mmol) of ytterbium oxide is added, and it is refluxed for 6 hours. After the complexing is completed, it is set at a pH of 7.4 with ammonia and chromatographed on silica gel (mobile solvent: dichloromethane/methanol/ammonia: 10/10/1). The fractions that contain the product are combined and absorptively precipitated with 10 g of ion exchanger (IR 267 H-form) for 2 hours and filtered off, then it is absorptively precipitated with 10 g of ion exchanger (IRA 67 OH-form) for 2 hours, filtered off, mixed with 2 g of activated carbon, heated for 2 hours to 60° C., filtered off and freeze-dried.

Yield: 11.1 g (62% of theory) of a colorless solid

Water content (Karl-Fischer): 6.5%

Elementary analysis (relative to the anhydrous substance):

Cld.: C, 32.68; H, 3.99; N, 10.39; I, 15.70; Yb, 21.40.

Fnd.: C, 32.81; H, 4.00; N, 10.36; I, 15.64; Yb, 21.27.

### Example 4

### 2,4,6-Triiodo-5-(3-aza-5-methyl-1,4-dioxopentane-1,5-diyl-{10-[1,4,7-tris-(carboxylatomethyl)-1,4,7,10-tetraazacyclododecanyl, Y-complex]})methylaminoisophthalic acid-N,N-bis-(3-aza-5-methyl-4-oxopentane-1,5-diyl-{10-[1,4,7-tris-(carboxylatomethyl)-1,4,7,10-tetraazacyclododecanyl, Y-complex]})amide

13.2 g (6.9 mmol) of 2,4,6-triiodo-5-(3-aza-5-methyl-1,4-dioxopentane-1,5-diyl-{10-[1,4,7-tris-(carboxymethyl)-1,4,7,10-tetraazacyclododecanyl]})methylaminoiso-phthalic acid-N,N-bis-(3-aza-5-methyl-4-oxopentane-1,5-diyl-{10-[1,4,7-tris-(carboxymethyl)-1,4,7,10-tetraazacyclododecanyl]})amide (title compound 1 g) is dissolved in 100 ml of water and acidified by adding 3 ml of acetic acid. 2.35 g (10.4 mmol) of yttrium oxide is added and refluxed for 6 hours. After the complexing is completed, it is set at a pH of 7.4 with ammonia and chromatographed on silica gel (mobile solvent: dichloromethane/methanol/ammonia: 10/10/1). The fractions that contain the product are combined and absorptively precipitated with 10 g of ion exchanger (IR 267 H-form) for 2 hours and filtered off, then absorptively precipitated with 10 g of ion exchanger (IRA 67 OH-form) for 2 hours, filtered off, mixed with 2 g of activated carbon, heated for 2 hours to 60° C., filtered off and freeze-dried.

Yield: 9.4 g (58% of theory) of a colorless solid

Water content (Karl-Fischer): 7.9%

Elementary analysis (relative to the anhydrous substance):

Cld.: C, 36.48; H, 4.45; N, 11.60; I, 17.52; Y, 12.27.

Fnd.: C, 36.61; H, 4.52; N, 11.65; I, 17.44; Y, 12.19.

The entire disclosures of all applications, patents and publications, cited herein and of corresponding German application No. 102004026103.2, filed May 25, 2004, and U.S. Provisional Application Ser. No. 60/575,417, filed Jun. 1, 2004, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Metal complexes of general formula I 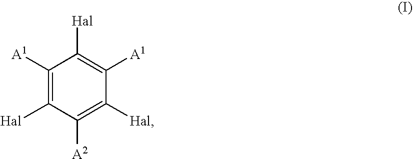
in which
Hal stands for bromine or iodine,
A¹ stands for the radical —CONH—(CH₂)₂—NH—CO—CH(CH₃)—K
A² stands for the radical —N(CH₃)—CO—CH₂—NH—CO—CH(CH₃)—K,
K stands for a macrocyclic compound of formula I_{A} 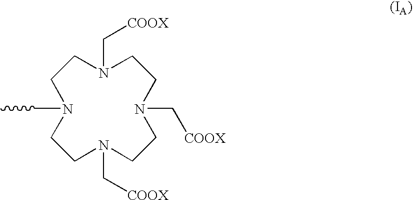
with X in the meaning of a hydrogen atom or a metal ion equivalent of atomic numbers 20-29, 39, 42, 44 or 57-83, provided that at least two X stand for metal ion equivalents and optionally present free carboxy groups optionally are present as salts of organic and/or inorganic bases or amino acids or amino acid amides.

2. Metal complexes according to claim 1, characterized in that X stands for a metal ion equivalent of atomic numbers 21-29, 42, 44, and 58-70.

3. Metal complexes according to claim 4, wherein X stands for a metal ion equivalent of the ions gadolinium(III), dysprosium(III), europium(III), iron(III) or manganese(II).

4. Pharmaceutical agent that contains at least one metal complex of general formula I according to claim 1, optionally with the additives that are commonly used in galenicals.

5. Use of at least one metal complex according to claim 1 for the production of agents for x-ray diagnosis.

6. Use of at least one metal complex according to claim 4 for the production of agents for MRT diagnosis.

7. Pharmaceutical agents that contain one metal complex each according to claim 1 in a molar ratio of 2000:1 to 1:1, preferably 49:1 to 4:1.

8. Pharmaceutical agent according to claim 6, wherein the metal complex(es) dissolved or suspended in water or or physiological salt solution is/are present at a concentration of 0.001 to 1 mol/l.

9. Use of at least one metal complex according to claim 1 for the production of agents for x-ray diagnosis and MR diagnosis of cerebral infarctions and tumors of the liver or space-occupying processes in the liver as well as tumors of the abdomen (including the kidneys) and the muscle-skeleton system and for the visualization of blood vessels after intraarterial or intravenous injection.

10. Process for the production of metal complexes of general formula I according to claim 1, wherein a triiodo- or tribromoaromatic compound of general formula II 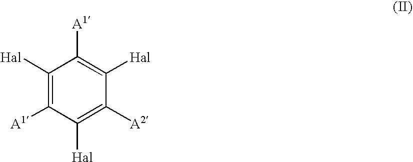
is reacted in a way that is known in the art with a macrocyclic compound of general formula III 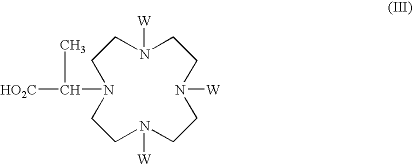
in which
W stands for a protective group, A^{1′} in the meaning of —CO—NH—(CH₁)₁—NH₂
and A^{2′} in the meaning of —N(CH₃)—C)—CH₂—NH₂
and then protective group W is removed and the radical CH₂COOX is introduced in a way that is known in the art and then reacted in a way that is known in the art with a metal oxide or metal salt of an element of atomic numbers 20-29, 39, 42, 44 or 57-83.

11. Process for the production of pharmaceutical agents according to claim 4, wherein the complex compound that is dissolved or suspended in water or physiological salt solution, optionally with the additives that are commonly used in galenicals, is brought into a suitable form for enteral or parenteral administration.
